(19) United States
(12) Plant Patent Application Publication
(10) Pub. No.: US 2015/0096091 P1
(43) Pub. Date: Apr. 2, 2015

(54) Sedum plant named 'Touchdown Teak'

(50) ;

(71) Applicant: Terra Nova Nurseries, Inc., Canby, OR (US)

(72) Inventor: Janet N. Egger, Wilsonville, OR (US)

(73) Assignee: Terra Nova Nurseries, Inc., Canby, OR (US)

(21) Appl. No.: 13/998,100

(22) Filed: Sep. 30, 2013

(51) Int. Cl. A01H 5/00

(52) U.S. Cl. PLT/479

(57) ABSTRACT

A new and distinct hybrid of Sedum plant substantially as shown and described, with dark red brown oval leaves, strong, dark red stems, multiple crowns, rose pink flowers, a medium short, upright habit, and excellent vigor.

### BOTANICAL DENOMINATION

Sedum telephium (Syn. Hylotelephium telephium)

### VARIETY DESIGNATION

‘Touchdown Teak’

### BACKGROUND OF THE INVENTION

The present invention relates to a new and distinct cultivar of Sedum telephium and given the cultivar name of ‘Touchdown Teak’. Sedum is in the family Crassulaceae. The new cultivar is part of a planned breeding program to make short, compact, multicrown plants. The new cultivar originated from an open pollinated cross with Sedum 31-7, a proprietary, unselected seedling, as the seed parent.

Compared to the seed parent, Sedum 31-7, the new cultivar, has a much better habit, creamy yellow flowers rather than light pink.

Compared to Sedum ‘Desert Black’, U.S. Plant patent application Ser. No. 13/815,310, the new cultivar has foliage that is dark red to dark red brown rather than dark brown.

This new and unique Sedum is distinguished by:

1. dark red brown oval leaves,

2. strong, dark red stems,

3. multiple crowns,

4. rose pink flowers,

5. a medium short, upright habit, and

6. excellent vigor.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows the foliage of a 2 year old Sedum ‘Touchdown Teak’ growing in a 2 gallon pot in early spring in Canby, Oreg.

FIG. 2 shows a 2 year old plant of Sedum ‘Touchdown Teak’ in the garden in May in Canby, Oreg.

FIG. 3 shows 2 year old plants in June in the garden in Canby, Oreg.

FIG. 4 shows a close up of the new cultivar in bloom in the trial field in mid September in Canby, Oreg.

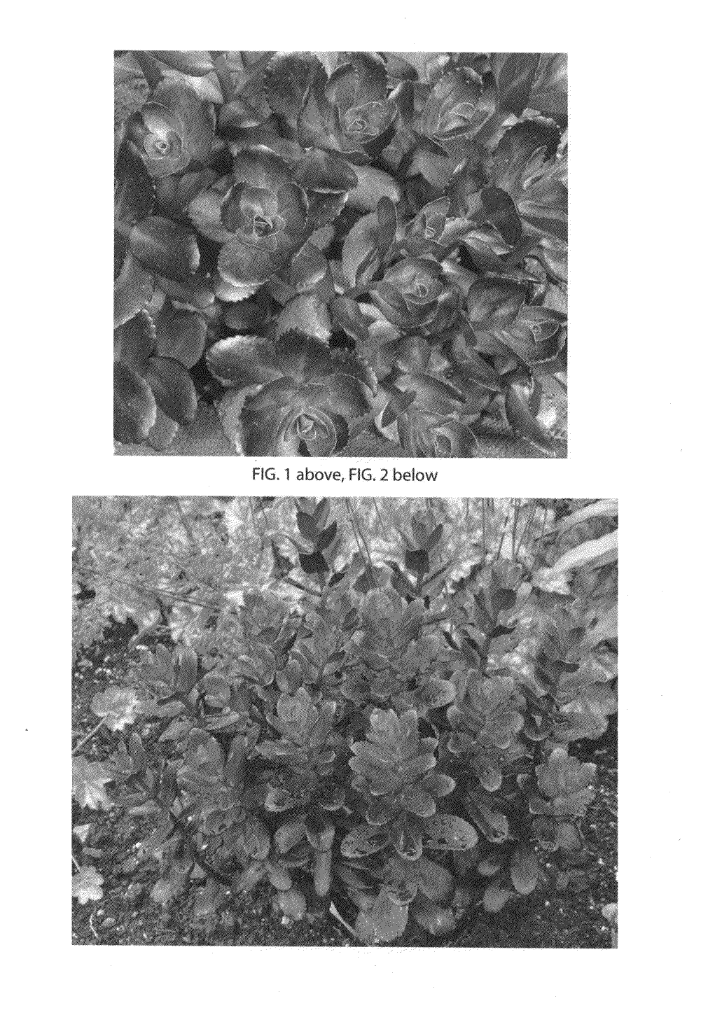

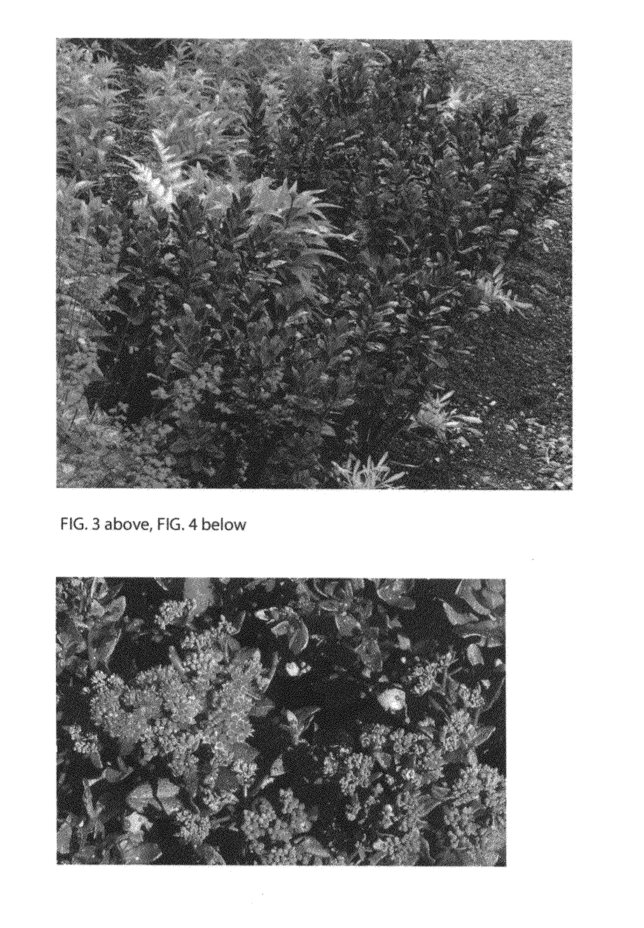

### DETAILED PLANT DESCRIPTION

The following is a detailed description of the new Sedum hybrid based on observations of 18 month old specimens grown in the trial bed in full sun under typical outdoor conditions in Canby, Oreg. Canby is Zone 8 on the USDA Hardiness map. Temperatures range from a high of 95° F. in August to 32° F. in January. Normal rainfall in Canby is 42.8 inches per year. The color descriptions are all based on The Royal Horticultural Society Colour Chart, 5^{th} edition.
Plant:
   Type.—herbaceous perennial.
   Hardiness.—USDA Zone 4 to 9.
   Size.—55 cm tall from the ground to the top the inflorescence and 45 cm wide.
   Habit.—low, clumping upright.
   Vigor.—excellent.
Stem:
   Number.—about 9 from base.
   Size.—50 cm long and 10 mm wide.
   Internode length.—2 cm to 3 cm long.
   Color.—Greyed Purple 187A.
   Texture.—succulent.
   Surface.—glabrous.
Leaf:
   Type.—simple.
   Arrangement.—opposite.
   Shape.—oblong.
   Apex.—obtuse.
   Base.—obtuse.
   Venation.—pinnate.
   Margins.—coarsely serrate.
   Blade size.—grows to 6.5 cm long and 3 cm wide.
   Surface.—glabrous on both sides.
   Texture.—thick, spongy, succulent.
   Petiole.—leaves sessile.
   Leaf color.—top and bottom side early spring Brown 200A with base Yellow Green 147A including vein; top and bottom side late spring, early summer between Black 203A and Greyed Purple 187A including vein; top and bottom side summer to fall Greyed Purple 187A including vein.
Inflorescence:
   Type.—terminal, corymbose, umbel-like.
   Number of flowers.—about 500 (increases with age).
   Size.—grows to 16.5 cm deep and 13 cm wide.
   Rachis.—grows to 14 cm tall and 5 mm wide, glabrous, Greyed Purple 187A.
   Pedicels.—2 mm long, 1 mm wide, glabrous, Greyed Purple N186C.
   Bloom period.—August in Canby, Oreg.
Flower bud:
   Size.—2.5 mm wide, 4 mm deep.
   Description.—ovoid.
   Color.—Greyed Purple 187D with tip Greyed Purple 187A.
Flower:
   Type.—actinomorphic.
   Shape.—stellate.
   Size.—4 mm deep and 5 mm wide.
   Corolla description.—4 mm deep and 5 mm wide, 5 petals, each lanceolate and 4.5 mm long and 1.5 mm wide, margins entire, tip acuminate, glabrous on both sides, top and bottom side Red Purple 62C on bottom half blending to Greyed Purple 187A at the tip.
   Calyx description.—1.3 mm deep and 2 mm wide, stellate, 5 lobes, divided to the base, margins entire, tip acute, glabrous, Greyed Purple N186C.
   Stamen description.—10, 3.5 mm long, filaments 3 mm long and Red Purple 62D, anthers Greyed Orange 173C, pollen Yellow Orange 18B.
   Pistil description.—2 mm wide and 3 mm deep, Red Purple 61A, with 5 separate prominent carpels, each 3 mm deep and 1 mm wide, Red Purple 61A on top half blending to White N155B on bottom half, style 1.5 mm long, style and stigma Red Purple 61A.
   Fragrance.—none.
   Lastingness.—each stem lasts up to a month.
Fruit:
   Type.—Ventrally dehiscent follicle, erect, attenuate.
   Fertility.—low.
   Size.—2.5 mm wide and 3 mm deep.
   Color.—Grey Brown N199B.
Seed: None seen
Disease and pest resistance or tolerance: Pest and disease resistance are typical for this type of Sedum. No known resistances.

I claim:

1. A new and distinct hybrid of Sedum plant as herein illustrated and described.
